(19) United States
(12) Patent Application Publication
(10) Pub. No.: US 2026/0183321 A1
(43) Pub. Date: Jul. 2, 2026

(54) TREATMENT OF CORONAVIRUS INFECTIONS WITH ANTIMICROBIAL PHOSPHOLIPID COMPOSITIONS

(71) Applicant: NATIONAL JEWISH HEALTH, Denver, CO (US)

(72) Inventors: Mari NAKAMURA, Denver, CO (US); Dennis VOELKER, Greenwood Village, CO (US)

(21) Appl. No.: 19/124,697

(22) PCT Filed: Oct. 31, 2023
(86) PCT No.: PCT/US2023/036380

§ 371 Date: Apr. 25, 2025

Related U.S. Application Data
(60) Provisional application No. 63/421,261, filed on Nov. 1, 2022.

(51) Int. Cl. A61K 31/683; A61K 9/00; A61K 9/08; A61P 31/14

(52) U.S. Cl. CPC A61K 31/683; A61K 9/0043; A61K 9/0073; A61K 9/08; A61P 31/14

(57) ABSTRACT

A method is provided for treating and/or inhibiting a coronavirus infection. The method includes administering to a subject who has, or is at risk of developing, said infection, an amount of an anionic lipid or related compound that is effective to inhibit said infection, wherein the anionic lipid has a hydrophobic portion, a negatively charged portion, and an uncharged, polar portion.

### CROSS-REFERENCE TO RELATED APPLICATIONS

This application claims the benefit of priority under 35 U.S.C. § 119(e) to U.S. Provisional Patent Application No. 63/421,261, filed Nov. 1, 2022. The entire disclosure of U.S. Provisional Patent Application No. 63/421,261 is incorporated herein by reference.

### FIELD OF THE DISCLOSURE

The present disclosure relates generally to phospholipid compositions, and more particularly to phospholipid compositions which may be utilized to treat coronavirus infections.

### BACKGROUND

Pulmonary surfactant was initially identified as a lipoprotein complex that reduces surface tension at the air-liquid interface of the alveolar compartment of the lung (Pattle, R. E. 1955. Nature 175:1125; Clements, J. A. 1957. Proc Soc Exp Biol Med 95:170). Pulmonary surfactant is synthesized and secreted by alveolar type II cells (King et al., 1973. Am J Physiol 224:788). Approximately 10% of the surfactant consists of proteins, including the hydrophilic surfactant proteins A and D (SP-A and SP-D), and the hydrophobic proteins, SP-B and SP-C (Kuroki and Voelker. 1994. J. Biol. Chem. 269:25943). SP-A and SP-D are now recognized to play important roles in innate immunity (Sano and Kuroki. 2005. Mol Immunol 42:279). SP-A and SP-D directly interact with various microorganisms and pathogen-derived components (Lawson and Reid. 2000. Immunol Rev 173:66). Moreover, by associating with cell surface pattern-recognition receptors, SP-A and SP-D regulate inflammatory cellular responses such as the release of lipopolysaccharide (LPS)-induced proinflammatory cytokines (Sano et al., 1999. J. Immunol. 163:387). LPS, derived from Gram-negative bacteria, is a potent stimulator of inflammation (O'Brien et al., 1980. J Immunol 124:20; Ulevitch and Tobias. 1995. Annu Rev Immunol 13:437). LPS molecules are engaged by the plasma LPS binding protein (LBP) (Wright et al., 1990. Science 249:1431) and transferred to CD14, a glycosylphosphatidylinisitol (GPI)-anchored protein, abundantly expressed on macrophages. LPS responses are dependent on the peripherally associated plasma membrane protein MD-2 (Nagai et al. 2002. Nat Immunol 3:667). and the membrane-spanning complex formed by toll-like receptor (TLR) 4 (Poltorak et al., 1998. Science 282:2085), through which signaling is propagated. TLRs activate four intracellular protein kinase cascades, the IB kinase (IKK)/NF-kB transcription factor cascade, the extracellular signal-regulated kinase (ERK), c-Jun NH2-terminal kinase (INK) and p38 mitogen-activated protein kinase (MAPK) cascades, leading to the induction of many key cytokine genes that are essential for the innate immune response (Takeda et al., 2003. Annu Rev Immunol 21:335; Medzhitov, R. 2001. Nat Rev Immunol 1:135; Barton and Medzhitov. 2003. Science 300:1524). At least one important function of SP-A and SP-D is to suppress the inflammatory response of the lung to LPS.

Pulmonary surfactant consists of approximately 90% by weight of lipids. Although the lipid composition varies in different species, its major component is phosphatidylcholine (PC) (70-80%). Nearly 80% of the PC is disaturated, consisting primarily of dipalmitoyl-phosphatidylcholine (DPPC). In addition, pulmonary surfactant contains variable amounts of phosphatidylglycerol (PG) (7-18%), phosphatidylinositol (PI) (2-4%) and phosphatidylethanolamine (PE) (2-3%) (Veldhuizen et al. 1998. Biochem Biophys Acta 1408:90). In contrast to PC, more than 50% of PG is unsaturated in many species, and in humans, there is little or no disaturated PG (Schmidt et al., 2002. Am J Physiol Lung Cell Mol Physiol 283:1079; Wright et al., 2000. J Appl Physiol 89:1283).

Previous work has provided some evidence that specific phospholipids may modulate inflammation. Oxidized phospholipid inhibits LPS-induced inflammatory responses in human umbilical-vein endothelial cells (Bochkov et al., 2002. Nature 419:77). Dioleoyl-phosphatidylglycerol (DOPG) inhibits phospholipase A2 secretion via a downregulation of NF-kB activation in guinea pig macrophages (Wu et al. 2003. Am J Respir Crit Care Med 168:692). Treponemal membrane phosphatidylglycerol inhibits LPS-induced immune responses from macrophages by inhibiting the binding of biotinylated LPS to LBP and blocking the binding of soluble CD14 (sCD14) to LPS (Hashimoto et al., 2003. J Biol Chem 278:44205). Cardiolipin, PG and PI exhibit an inhibitory effect on LPS-induced TNF-α production by human macrophages, possibly by a blockade of the binding of LPS aggregates to LBP (Mueller et al., 2005. J Immunol 172:1091). However, very few reports have focused on the potential anti-inflammatory roles of surfactant phospholipids on either alveolar or non-alveolar macrophages. Moreover, the relationship between surfactant phospholipids and CD14 or other pattern recognition receptors has not been clearly identified.

Various studies have made connections between surfactant PG content and disease. For example, in idiopathic pulmonary fibrosis patients, some groups reported decreased unsaturated PG in surfactant (Veldhuizen et al., 1998, Biochem Biophys Acta 1408:90; Honda et al., 1988, Lung 166:293; and Saydain et al., 2002, Am J Resp Crit Care Med 166:839). In another disease, acute respiratory distress syndrome (ARDS), Schmidt et. al. have reported significant reduction in the unsaturated PG recovered in BALF (Schmidt et al., 2001, Am J Respir Crit Care Med 163:95). The issues of cause and effect in the above diseases remain unclear.

LPS is a major cause of acute lung injury (ALI) and ARDS (Atabai and Matthay. 2002. Thorax 2002; Rubenfeld et al., 2005. N Engl J Med 353:1685). ALI/ARDS is a life-threatening condition in which inflammation of the lungs and accumulation of fluid in the alveoli leads to low blood oxygen levels. Over a period of 25 years the annual incidence of ALI/ARDS is 335,000, with 147,000 deaths per year. The most common risk factor for ALI was severe sepsis with a suspected pulmonary source (46%), followed by severe sepsis with a suspected nonpulmonary source (33%).

Severe acute respiratory syndrome coronavirus 2 (SARS-CoV-2) has infected 33 million people and caused 592,000 deaths in the US, as of June 2021 (1 Novel Coronavirus Reports: Centers for Disease Control and Prevention; 2021. Available from: cdc.gov/mmwr/Novel_Coronavirus_Reports.html). The pathogenicity of the viral disease (COVID-19) remains to be fully elucidated. Genetic variants of SARS-CoV-2 have been emerging and expanding (1 Novel Coronavirus Reports). There are growing concerns that these variants might contribute to an increase in transmissibility and result in more severe disease (1 Novel Coronavirus Reports). Chronic obstructive pulmonary disease (COPD) is a major medical complication associated with increased mortality from SARS-CoV-2 infections (2, 3 2. Leung J M, et al. COVID-19 and COPD. Eur Respir J. 2020; 56(2). Epub 2020/08/21. doi: 10.1183/13993003.02108-2020. PubMed PMID: 32817205; PMCID: PMC7424116 M.; Halpin DMG, et al. Global Strategy for the Diagnosis, Management, and Prevention of Chronic Obstructive Pulmonary Disease 2021 Update Report. Available from: goldcopd.org/wp-content/uploads/2020/11/GOLD-REPORT-2021-v1.1-25Nov20_WMV.pdf). Currently few effective therapies are available for COVID19.

Given the severity of symptoms associated with many inflammatory conditions and viral causing conditions, including those affecting the respiratory system, there is a continued need for agents useful in controlling inflammation and thereby preventing and/or treating conditions or diseases associated with inflammation.

### SUMMARY

In one aspect, a method is provided for treating or inhibiting a coronavirus infection. The method comprises administering to a subject who has, or is at risk of developing, said infection, an amount of at least one anionic lipid or related compound, wherein the amount of the anionic lipid or related compound is effective to prevent or inhibit said coronavirus infection, and wherein the anionic lipid has a hydrophobic portion, a negatively charged portion, and an uncharged, polar portion.

In some embodiments, the anionic lipid or related compound is selected from the group consisting of: unsaturated phosphatidylglycerol, unsaturated phosphatidylinositol, saturated short chain phosphatidylglycerol, saturated short chain phosphatidylinositol, anionic sphingolipid, anionic glycerolipid, unsaturated lyso-phosphatidylglycerol, saturated lyso-phosphatidylglycerol, unsaturated lyso-phosphatidylinositol, and saturated lyso-phosphatidylinositol, or a derivative thereof.

In some embodiments, the anionic lipid is selected from the group consisting of an unsaturated phosphatidylglycerol, an unsaturated phosphatidylinositol, a saturated short chain phosphatidylglycerol, and a saturated short chain phosphatidylinositol, or a derivative of the anionic lipid.

In some embodiments, the anionic lipid or related compound is unsaturated phosphatidylglycerol, or a derivative thereof.

In some embodiments, the anionic lipid or related compound is palmitoyl-oleoyl-phosphatidylglycerol (POPG), or a derivative thereof.

In some embodiments, the anionic lipid or related compound is unsaturated phosphatidylinositol (PI), or a derivative thereof.

In some embodiments, the coronavirus infection is associated with at least one toll-like receptor (TLR) selected from the group consisting of TLR1, TLR2, TLR3, TLR4, TLR6, TLR7, TLR8, TLR9, and TLR10.

In some embodiments, the anionic lipid or related compound is administered as a composition comprising a homogeneous lipid preparation of the anionic lipid or related compound.

In some embodiments, the anionic lipid or related compound is administered as a composition comprising a preparation of randomly mixed surfactant lipids combined with a homogeneous lipid preparation of the anionic lipid or related compound.

In some embodiments, the anionic lipid or related compound is administered as a preparation of randomly mixed surfactant lipids, wherein the anionic lipid or related compound comprises at least about 50% of the total lipids in said randomly mixed surfactant lipids.

In some embodiments, the anionic lipid or related compound is administered to the respiratory tract of the individual.

In some embodiments, the anionic lipid is administered to the upper respiratory tract of the subject intranasally or lower respiratory tract via inhalation.

In some embodiments, the anionic lipid is administered intranasally as a formulation containing an emulsifier, a stabilizer and a preservative.

In some embodiments, the coronavirus infection involves at least one virus selected from the group consisting of SARS-CoV-2, SARS-CoV, MERS-CoV and variants thereof.

In some embodiments the coronavirus infection involves SARS-CoV-2.

### BRIEF DESCRIPTION OF THE DRAWINGS

FIGS. 1A and 1B show that POPG and PI inhibit SARS-CoV-2 replication in VeroE6 cells at 24 hrs. VeroE6 cells were infected with SARS-CoV-2 at an MOI=0.01 in either the absence of lipids, or presence of POPG, or PI, or POPC, at concentrations of 250 μg/ml-1000 μg/ml (SARS-CoV-2+POPG, SARS-CoV-2+PI) or the control lipid phosphatidylcholine (PC), at various concentrations (250 μg-1000 μg/ml) (SARS-CoV-2+PC). Cells were preincubated with lipids 30 mins before viral inoculation. Culture media was harvested at 24 hr and processed for quantitative plaque assay for SARS-CoV-2 (FIG. 1A) and for viral yield as a function of phospholipid concentration (FIG. 1). The data are shown as mean+/−SE for three independent experiments in log₁₀ scale. is a graph of viral yield as a function of phospholipid concentration.

FIG. 2 shows that POPG and PI prevent cytopathic effect by SARS-CoV-2. Histological images of VeroE6 cells at 72 hrs after SARS-CoV-2 infection. Cells were either sham infected (CONL) or treated with lipids: POPG (1000 μg/ml), PI (500 μg/ml), and PC (2000 μg/ml). Cells were washed with PBS, fixed with 10% buffered formalin, and stained with Crystal violet. Scale bar in 50 μm.

FIGS. 3A, 3B, 3C, and 3D show that POPG and PI inhibit viral replication in differentiated bronchial (trachea) epithelial cells (FIGS. 3A and 3C) and nasal epithelial cells (FIGS. 3B and 3D) of control subjects at 48 hrs.

FIGS. 4A, 4B, 4C, and 4D show that POPG and PI inhibit replication in differentiated nasal epithelial cells of control subjects (FIGS. 4A and 4C) and severe asthmatics (FIGS. 4B and 4D) at 48 hrs.

FIG. 5 shows that PI and POPG bind to SARS-CoV-2 S1 ECD protein with weak affinity and non-specific manner.

FIGS. 6A, 6B, 6C, and 6D show intranasal treatment with PI inhibits SARS-CoV-2 replication in an in vivo model in hamsters. Hamsters were challenged with SARS-CoV-2 (103 particles) using a 100 μl intranasal inoculum in either the absence, or presence of PI (2 mg/animal). Viral burdens were determined by plaque assays using pharyngeal swabs performed on days 1-3.

FIGS. 7A and 7B show the lung histology score using the in vivo hamster model used in FIGS. 6A-6D. FIG. 7A is at day 3 and FIG. 7B is at day 7.

FIGS. 8A and 8B show POPG and PI inhibit viral replication in differentiated trachea cells and nasal epithelial cells of control subjects at 48 hrs. Human primary airway epithelial cells were grown in air liquid interface (ALI) culture for 24 days and visualized with H&E staining (not shown). POPG and PI inhibited SARS-CoV-2 replication in differentiated bronchial (FIG. 8A) and nasal epithelial cells (FIG. 8B) in ALI. cultures Bronchial or nasal epithelial cells were infected with SARS-CoV-2 at an MOI=0.2 in either the absence of lipids, or presence of 5 mg/ml POPG, or 2 mg/ml PI (SARS-CoV-2+POPG, SARS-CoV-2+PI), or the control lipid, phosphatidylcholine (PC), at 10 mg/ml (SARS-CoV-2+PC) in apical media. At 48 hrs after infection, SARS-CoV-2 mRNA expression was determined by qRT-PCR. Numerical values are means±SD. * Indicates, p<0.05, §§ indicates, p<0.001 (statistical analysis by single Anova and paired t-test), respectively.

FIG. 9 shows that POPG and PI inhibit SARS-CoV-2 variant, B.1.351 replication in bronchial epithelial cells from a healthy control subject in air liquid interface (ALI) cultures. POPG and PI inhibited SARS-CoV-2 replication in differentiated bronchial epithelial cells grown in ALI. Bronchial epithelial cells were infected with SARS-CoV-2 at an MOI=0.02 in either the absence of lipids or presence of 5 mg/ml POPG (B1.351+POPG) or 2 mg/ml PI (B1.351+PI), or the control lipid, phosphatidylcholine (PC), 10 mg/ml (B1.351+PC) in apical media. B1.351 replication was determined.

FIG. 10 shows POPG and PI inhibit SARS-CoV-2 replication in differentiated nasal epithelial cells in ALI cultures from a patient with COPD. POPG and PI inhibited SARS-CoV-2 replication in differentiated nasal epithelial cells from a patient with COPD. Nasal epithelial cells were infected with SARS-CoV-2 in either the absence of lipids, or presence of 5 mg/ml POPG (B1.351+POPG) or 2 mg/ml of PI (B1.351+PI) or the control lipid, phosphatidylcholine (PC), 10 mg/ml (B1.351+PC) in apical media. B1.351 replication was determined by qRT-PCR at 48 hrs post-infection.

FIGS. 11A and 11B show POPG or PI does not produce pleiotropic inhibition of metabolism in primary human bronchial epithelial cells in ALI cultures. Bronchial epithelia cells were incubated for 64 h with 3H-leucine in either the presence, or absence of 5 mg/mL of POPG; or 2 mg/ml of PI in only the apical media. 3H-intracellular protein synthesis (FIG. 11A), and 3H secreted protein in apical media (FIG. 11B) are shown. Values shown are means±SD for three independent experiments using 3 different healthy control subjects.

FIGS. 12A and 12B show lipids attenuate SARS-CoV-2 variant replications in differentiated human primary bronchial epithelial cells from healthy control subjects. Differentiated human primary bronchial epithelial cells from healthy control subjects (subject numbers were n=4) were challenged with SARS-CoV-2 variants (B.1.617.2 (delta variant) and B1.1.529 (omicron variant)) at multiplicity of infection (m.o.i.)=0.1. Groups: virus alone (B.1.617.2 or B.1.1.529), Virus+prophylactic lipid treatment: 10 mg/ml POPG (B.1.617.2 or B.1.1.529+POPG), 4 mg/ml PI (B.617.2 or B.1.1.529+PI) in apical media. FIG. 12A: B.1.617.2 (delta variant) replication was inhibited by POPG and PI treatments at 48 hrs after infection FIG. 12B: POPG and PI inhibited B.1.1.529 replication (Omicron variant) at 48 Hr. §§ indicates p<0.001 and § indicates p<0.01, respectively. The data was shown as mean±SD.

### DETAILED DESCRIPTION

Primary human airway epithelial cells, nasal epithelial cells, and bronchial epithelial cells all highly express angiotensin-converting enzyme 2 (ACE2), the major receptor for SARS-CoV-2, and also express the transmembrane serine protease (TMPRSS2) that is crucial for SARS-CoV-2 cell entry (Sungnak W, et al., Network HCALB. SARS-CoV-2 entry factors are highly expressed in nasal epithelial cells together with innate immune genes. Nat Med. 2020; 26(5):681-7; Hoffmann M, et al. SARS-CoV-2 Cell Entry Depends on ACE2 and TMPRSS2 and Is Blocked by a Clinically Proven Protease Inhibitor. Cell. 2020; 181(2):271-80 e8.). These features are especially true for nasal epithelial cells, which serve as a gateway cell population for virus entry and subsequent propagation to eventually expand the infection into the gas exchange regions of the lungs. The pulmonary surfactant system is a complex of secreted lipids and proteins that plays central role in regulating innate immunity within the lung (Wright J R. Immunomodulatory functions of surfactant. Physiol Rev. 1997; 77(4):931-62). The inventors have previously identified the minor pulmonary surfactant phospholipids, POPG, and PI, as critical regulators of Toll-like receptor mediated inflammatory processes, which significantly contribute to the cytokine storm (Voelker D R, Numata M. Phospholipid regulation of innate immunity and respiratory viral infection. J Biol Chem. 2019; 294(12):4282-9) that results from respiratory viral infection (Voelker D R, J Biol Chem. 2019; 294(12):4282-9).

It has now been found that particular surfactant phospholipids, and particularly, anionic phospholipids, may be potent inhibitors of coronavirus infections, including those associated with SARS-CoV-2 (including variants B1.1.7 and B.1.351), SARS-CoV and MERS-CoV and their variants, and may also alleviate the symptoms of such infections. Specifically, it has been found that unsaturated phosphatidylglycerols (PGs or PtdGro), including, but not limited to palmitoyl-oleoyl-phosphatidylglycerol (POPG), unsaturated phosphatidylinositols (PIs or PtdIns), and selected short chain saturated phospholipids, including, but not limited to, short chain saturated PGs (e.g., dimyristoyl-phosphatidylglycerol (DMPG) or 14:0/14:0-PtdGro), may be potent inhibitors of such coronavirus infections, and may alleviate the symptoms of such infections.

In addition, it has been found that, in addition to the above-described lipids, any anionic lipid may be useful in treating such coronavirus infections (or alleviating the symptoms of such infections) if it has a hydrophobic portion; a negatively charged portion; and an uncharged, polar portion. Such lipids include, but are not limited to, the above-mentioned phospholipids, anionic sphingolipids, anionic glycerolipids (e.g., anionic diglycerides from plants, such as SQV-diglycerides). In addition, it has been further found that compounds closely related to unsaturated PG and unsaturated PI, and particularly lyso-PG and lyso-PI, including, but not limited to, saturated or unsaturated lyso-PG, and saturated or unsaturated lyso-PI, may be utilized for the prevention and/or inhibition of such coronavirus infections or the symptoms thereof.

Without wishing to be bound by theory, it is believed that the inhibitory activity of the lipids and compounds disclosed herein may be attributed to activation of the specific toll receptors, TLR1, TLR2, TLR3, TLR4, TLR6, TLR7, TLR8, TLR9 and TLR10. Accordingly, homogeneous preparations of these anionic lipids and related compounds, as well as various compositions comprising these anionic lipids and related compounds, may be utilized to prevent or treat the foregoing coronavirus infections or inflammation associated with these infections, especially inflammation associated with the activation of TLR1, TLR2, TLR3, TLR4, TLR6, TLR7, TLR8, TLR9, TLR10.

In some embodiments of the compositions and methodologies disclosed herein, substantially homogeneous preparations of the anionic lipids and/or related compounds (described above and in more detail below) are provided, which may be utilized in any of the preventative or therapeutic methods described herein alone, or by admixture (combination, directed mixing) with other lipids, including surfactant preparations. In some embodiments, randomly mixed surfactant preparations are provided, wherein at least 50% of the total lipids in the preparations include one or more of the particular anionic phospholipids or lipids or related compounds of the type disclosed herein.

Preferred embodiments of the compositions disclosed herein comprise an effective amount of at least one anionic lipid having at least a hydrophobic portion; a negatively charged portion; and an uncharged, polar portion. Such anionic lipids may therefore include, but are not limited to, unsaturated phosphatidylglycerols, unsaturated phosphatidylinositols, saturated short chain phosphatidylglycerols, saturated short chain phosphatidylinositols, and derivatives of any of such phospholipids (e.g., polyethylene glycol (PEG) conjugates of these phospholipids), as well as anionic sphingolipids, anionic glycerolipids (anionic diglycerides, such as SQV-diglyceride), and any derivatives of such lipids. Preferred phospholipids include, but are not limited to, unsaturated phosphatidylglycerols, unsaturated phosphatidylinositols, palmitoyl-oleoyl-phosphatidylglycerol (POPG), and dimyristoyl-phosphatidylglycerol (DMPG). In one preferred embodiment, the phospholipids are selected from palmitoyl-oleoyl-phosphatidylglycerol (POPG) and/or phosphatidylinositol (PI) and/or derivatives thereof.

In some embodiments, compounds closely related to unsaturated PG and unsaturated PI (and particularly, lyso-PG and lyso-PI) may be utilized as inhibitors of inflammation attendant to a coronavirus infection, and particularly, as antagonists of TLRs. Because lyso-PG and lyso-PI have much higher water solubility and form micellar rather than bilayer structures, they may have greater access from the bulk solution to the TLRs. Thus, saturated or unsaturated lyso-PG, and saturated or unsaturated lyso-PI may be useful for the prevention and treatment of inflammation attendant to a coronavirus infection, and as antagonists of the activation of TLRs 1, 2, 3, 4, 6, 7, 8, 9 and in some embodiments, TLR10. General reference to “related compounds” with respect to the anionic lipids of the invention, refers to these lyso-PG and lyso-PI compounds, or other similar compounds.

Phosphatidylglycerol (PG) is a ubiquitous phospholipid that is a major component of bacterial cell membranes and a lesser component of animal and plant cell membranes. In animal cells, PG may serve primarily as a precursor for diphosphatidylglycerol (cardiolipin). PG is the second most abundant phospholipid in lung surfactant in most animal species. A particularly useful PG in the present invention is palmitoyl-oleoyl-phosphatidylglycerol (POPG).

Phosphatidylinositol (PI) is a key membrane constituent and is a participant in essential metabolic processes in all plants and animals (and in some bacteria (Actinomycetes)), both directly and via a number of metabolites. It is an acidic (anionic) phospholipid that in essence consists of a phosphatidic acid backbone, linked via the phosphate group to inositol (hexahydroxycyclohexane). In most organisms, the stereochemical form of the last is myo-D-inositol (with one axial hydroxyl in position 2 with the remainder equatorial), although other forms (scyllo- and chiro-) have been found on occasion in plants. PI is formed biosynthetically from the precursor CDP-diacylglycerol by reaction with inositol, catalysed by the enzyme CDP-diacylglycerol inositol phosphatidyltransferase.

Unsaturated PGs and PIs are defined herein as any PG or PI with one or more double bonds in the fatty acid chain.

Saturated PGs or PIs are defined herein as any PG or PI without a double bond (i.e., the chains are fully saturated with hydrogens). Preferred saturated short chain PGs or PIs useful in the present invention includes any saturated 14 carbon or shorter PG or PI with anti-inflammatory properties as described herein. A particularly preferred saturated short chain PG includes, but is not limited to, dimyristoyl-phosphatidylglycerol (DMPG).

As the term is used herein, compositions containing an “effective amount” of an anionic lipid or related compound of the type described herein contain an amount of the specific anionic lipid or related compound effective to inhibit a coronavirus-associated inflammatory process in vitro or in vivo, or to inhibit a coronavirus infection in vitro or in vivo, as measured by any suitable technique for measuring such activity. Effective amounts of anionic lipids or related compounds are described in greater detail below.

In some embodiments of the compositions and methodologies described herein, the anionic lipids or related compounds may be provided in a homogeneous lipid preparation comprising, consisting essentially of, or consisting of one or more of the anionic lipids or related compounds described herein, and/or derivatives of any of such anionic lipids or related compounds. In some embodiments, any of the above-described lipid preparations may further comprise any other lipid or lipid derivative that is useful in a surfactant preparation, useful in a therapeutic preparation, and/or useful for stabilizing the bilayer of lipids in a lipid preparation and/or decreasing leakage of encapsulated material. In some embodiments, any of the lipid preparations described herein may further comprise antioxidants, which may be useful for inhibiting oxidation of the lipids in lipid preparation.

The lipid preparations described herein may include any stabilized form of lipid that would be useful in the methodologies disclosed herein. These lipids may be stabilized by proteins or other suitable compounds. Examples of such lipid preparations may include, but are not limited to, liposomes and protein-stabilized lipid forms (e.g., non-liposomal lipids stabilized by the use of a lipoprotein, e.g., see NANODISC™, Nanodisc, Inc.). As used herein, the term “liposome” (also referred to as a liposomal preparation or liposomal composition) is a spherical, microscopic artificial membrane vesicle consisting of an aqueous core enclosed in one or more phospholipid layers. Liposomes may also be generally defined as self closed spherical particles with one or several lipid membranes. Liposomes may include naturally-derived phospholipids with mixed fatty acid chains or prepared from synthetic lipids with well-defined lipid chains. Depending on the number of the membranes and size of the vesicles, liposomes are considered to be large multilamellar vesicles (LMV) with sizes up to 500 nm, small unilamellar vesicles (SUV) with sizes <100 nm, and large unilamellar vesicles (LUV) with sizes >100 nm. Liposomes and liposome preparation methods are well known in the art. A stabilized lipid, such as a protein- or lipoprotein-stabilized lipid, may be prepared using any method known in the art.

In some embodiments, the lipid in the lipid preparation is composed of pure unsaturated PG, pure unsaturated PI, pure saturated short chain PG, pure saturated short chain PI, pure anionic sphingolipid, pure anionic glycerolipid, pure unsaturated lyso-PG, pure saturated lyso-PG, pure unsaturated lyso-PI, pure saturated lyso-PI, or any combination thereof. In some embodiments, the lipid in the lipid preparation includes pure palmitoyl-oleoyl-phosphatidylglycerol (POPG), dimyristoyl-phosphatidylglycerol (DMPG), pure unsaturated PI, pure unsaturated PG, or any combinations thereof. Similarly, lipid preparations may include any of these anionic lipids or related compounds, in combination with one or more different phospholipids and/or other lipid(s) and/or related compounds.

Preferred compositions for use in the methodologies disclosed herein provide a suitable amount of anionic lipids or related compounds to provide a therapeutic or anti-inflammatory or anti-pathogen (e.g., anti-viral) effect when administered to an individual who is suffering from a coronavirus infection, or who may be exposed to a coronavirus.

Various compositions of the anionic lipids or related compounds described herein may be utilized in the methodologies disclosed herein. In some embodiments, the anionic lipid may be formulated as a homogeneous preparation consisting of the anionic lipid or related compound. As used herein, a “homogeneous” lipid preparation consisting of a specified anionic lipid or related compound or combination of specified anionic lipids or related compounds, means that the lipid preparation (e.g., the lipid vesicles or smaller portions) contains only the specified anionic lipid or related compound or a combination of specified anionic lipids or related compounds (e.g., a pure preparation of the specified phospholipid(s)), and is substantially or completely free of other phospholipids or other lipids. A homogeneous preparation of a specified anionic lipid or related compound can contain other non-lipid agents, if desired, such as antioxidants, a targeting moiety (described below), or another therapeutic agent (e.g., a protein, and antibody, a small molecule or drug). A homogeneous lipid preparation of the type disclosed herein may be provided alone or with a pharmaceutically acceptable carrier, including an excipient or buffer, or in a composition with other agents or lipid preparations.

Some embodiments of the methodologies disclosed herein may utilize xylitol-headgroup lipid analogs. Suitable compositions of this type are described, for example, in U.S. US2020/0009165 (Voelker), which is incorporated herein by reference in its entirety.

In some embodiments of the methodologies disclosed herein, a homogeneous lipid preparation of an anionic lipid or related compound may be administered in the absence of any other lipids, although in other embodiments, the additive effects of other lipids, such as other lipids contained in surfactant, may be desirable and useful. In these alternate embodiments, compositions may be utilized that allow for the provision of such additional lipids and/or combinations of lipids, without losing the effectiveness of the particular anionic lipids or related compounds described herein.

Some embodiments of the methodologies disclosed herein may utilize a homogeneous lipid preparation of the anionic lipid(s) or related compound(s) of the type disclosed herein, and at least one additional agent. The additional agent may include any suitable pharmaceutical carrier, or an additional agent such as, for example, the treatment of inflammation or pathogen infection (e.g., an anti-viral or anti-bacterial agent).

Suitable anti-inflammatory agents which may be utilized in the compositions and methodologies disclosed herein include, but are not limited to, cytokine inhibitors, chemokine inhibitors, chemoattractant inhibitors, Cox inhibitors, leukotiene receptor antagonists, leukotriene synthesis inhibitors, inhibitors of the p38 MAP kinase pathway, glucocorticoids. More specifically, anti-inflammatory compounds may include, but are not limited to, any suitable inhibitor of eicosanoid synthesis and release, including any Cox-2 inhibitor; Cox-1 inhibitors; inhibitors of some certain prostaglandins (prostaglandin E(2); PGD(2)), inhibitors of certain leukotrienes (LTB4); classes of antibiotics with known direct or indirect anti-inflammatory effects, including macrolides (e.g. azithromycin) and fluoroquinolones (e.g., levofloxacin; moxifloxacin; gatifloxacin); inhibitors of p38 MAP kinase; inhibitors of the function of pro-inflammatory cytokines and chemokines, including antagonists of tumor necrosis factor (TNF), antagonists of interleukin-8 (IL-8); transforming growth factor beta (TGF-beta), β-agonists (long or short acting), antihistamines, phosphodiesterase inhibitors, corticosteroids, and other agents.

As used herein, the term “pharmaceutically acceptable carrier” includes pharmaceutically acceptable excipients and/or pharmaceutically acceptable delivery vehicles, which are suitable for use in the administration of a preparation, formulation or composition, including a liposomal composition or preparation, to a suitable in vivo site. A suitable in vivo site is preferably any site wherein inflammation or infection by a coronavirus is occurring or is expected to occur. Preferred pharmaceutically acceptable carriers are capable of maintaining the formulation in a form that, upon arrival of the formulation at the target site in a subject (such as, for example, in the lung tissues of the subject), the formulation is capable of acting at the site, preferably resulting in a beneficial or therapeutic benefit to the subject (here, it is noted that the subject is preferably human, but may be an animal subject in veterinary or research applications of the methodologies disclosed herein). A delivery vehicle for a protein or agent may include the lipid preparation itself if another agent is included, although in many embodiments, the lipid preparation is also a therapeutic agent as described herein (e.g., the lipid preparation can serve one or both functions).

Suitable excipients which may be useful in the compositions and methodologies disclosed herein include excipients or formularies that transport or help transport, but do not specifically target, a composition or formulation to a cell or tissue (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers may contain suitable auxiliary substances required to approximate the physiological conditions of the recipient as, for example, by enhancing chemical stability and isotonicity. Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Formulations of the present invention can be sterilized by conventional methods and/or lyophilized.

Lipid preparations of the type disclosed herein may be modified to target to a particular site in a subject, thereby targeting and making use of the anionic lipids or related compounds and any agents carried by the lipid preparation at that site. Suitable modifications include manipulating the chemical formula of the lipid preparation and/or introducing into the lipid preparation a targeting agent capable of specifically targeting the lipid preparation to a preferred site such as, for example, a preferred cell type. Suitable targeting agents include ligands capable of selectively (i.e., specifically) binding another molecule at a particular site. Examples of such ligands include antibodies, antigens, receptors and receptor ligands.

In some embodiments, the composition utilized in the methodologies disclosed herein may comprise a preparation (e.g., a lipid preparation) of randomly mixed anionic lipids or related compounds (any combination), and preferably, randomly mixed surfactant phospholipids or lipids (e.g., any combination of lipids found in surfactant), combined with (added to, mixed gently with, in admixture with) a homogeneous lipid preparation of the anionic lipids or related compounds of the type disclosed herein. In such embodiments, the combining of the randomly mixed lipids with the homogeneous lipid preparation of the anionic lipids or related compounds may be performed in a manner that does not result in significant fusion and/or intermixing of lipids between the vesicle bilayers (e.g., between the randomly mixed lipid preparations and the pure or homogeneous lipid preparation of anionic lipids or related compounds). By producing a homogeneous preparation of the desired anionic lipids or related compounds and then adding it to another preparation of lipids, such as a randomized surfactant preparation, the biological activity of the anionic lipids or related compounds described herein (such as, for example, their anti-inflammatory or anti-viral activity) may be maintained. In embodiments of this type, it is preferred that the homogeneous lipid preparations of the anionic lipids or related compounds comprise at least 1% of the total lipids in the composition (e.g., the total lipids being those present in the homogeneous preparation and the added randomly mixed surfactant preparation), or at least 5%, or at least 10%, or at least 15%, or at least 20%, or at least 25%, or at least 30%, or at least 35%, or at least 40%, or at least 45%, or at least 50%, of the total lipids in the composition.

In other embodiments of the methodologies disclosed herein, a preparation of randomly mixed lipids is provided, and preferably a preparation of randomly mixed surfactant lipids and phospholipids, wherein the preparation contains one or more anionic lipids or related compounds useful in the present invention as described above. In such embodiments, the anionic lipid(s) or related compounds preferably comprise at least about 30% of the total lipids in the randomly mixed surfactant lipids, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, of the total lipids in the randomly mixed surfactant lipids (or any amount between at least 30% and 100%, in whole number increments, e.g., 30%, 31%, 32%, etc.).

Preparations of randomly mixed lipids, and particularly, randomly mixed surfactant lipids, may be made using techniques known in the art and are also available commercially (e.g., see EXOSURF® (Wellcome, USA, an artificial surfactant preparation); ALVEOFACT® (Thomae, Germany, prepared from bovine BAL); CUROSURF® (Chiesi, Italy, prepared from minced porcine or bovine lung tissue) or SURVANTA® (Abbott, USA, prepared from minced porcine or bovine lung tissue)). Lung surfactant is a complex mixture of various phospholipids, neutral lipids and apoproteins (Doles, Ann Rev Med 1989; 40: 431-446; Jobe, N Engl J Med 1993; 328: 861-868; Tegtmeyer et al., Eur Respir J, 1996, 9, 752-757). Surfactant replacement therapy has proven to be beneficial for the treatment of the neonatal respiratory distress syndrome (Jobe, supra), and is also considered as a therapeutic option for term infants and adults with acute respiratory failure (Lewis and Jobe, Am Rev Respir Dis 1993; 147:216-233). Accordingly, surfactant lipid preparations are widely available and well known to those of skill in the art. It is believed that the addition of the homogeneous lipid preparations of anionic lipids and related compounds described herein to such preparations will significantly enhance the use of such commercial preparations or other surfactant preparations in the prevention and treatment of a variety of conditions, including those attendant to coronavirus infections.

The total concentration of lipids to be delivered to a human subject (and in particular, to the lungs of the subject) according to the present invention may range from about 5 mol to about 1 mmol, including any amount between, in increments of 1 mol. In one aspect, the amount delivered is from about 40 mol to about 800 M, although one skilled in the art can readily determine the appropriate amount to be delivered. By way of example but not limitation, in one embodiment, the lipid preparation comprising a given anionic lipid (e.g., unsaturated PG) is delivered in an amount suitable to replace all resident lung PG). The estimated amount of unsaturated PG in the lung is approximately 400 μmole in the entire adult human lung residing in the alveolar compartment exclusive of the tissue. If the lipid preparation is to replace all resident lung PG, then 40 μmol/mlx 10 ml would be sufficient.

In preferred embodiments of the methodologies disclosed herein, the anionic lipid(s) and/or related compound(s) are administered to the lung in an amount delivered that is equivalent to between about 10% of the total resident amount of the same or similar lipid, to about 200% of the total resident amount. Accordingly, from a lipid preparation that is 40 ρmol of the lipid or compound of the invention per ml of lipid preparation, the individual would receive between about 1 ml and 20 ml delivered in an aqueous suspension down the trachea, for delivery to the lungs.

In some embodiments, the lipid preparation used in the methodologies disclosed herein may be complexed with another agent, such as a protein or a small molecule (drug), wherein the other agent is also useful for inhibiting or preventing inflammation or infection by a pathogen (e.g., a virus) in an individual. Methods of encapsulating or complexing proteins and other agents with lipids such as liposomes and protein-stabilized lipids are known in the art. The encapsulation efficiency of proteins by lipid preparations generally depends on interaction between the protein and the lipid bilayer or micelle. The protein entrapment may be increased by manipulation of the lipid preparation, or by increasing the lipid concentration, in order to favor electrostatic interactions, while monitoring the ionic strength of the protein solution (Colletier et al., BMC Biotechnology 2002, 2:9). Preferably, the amount of a protein complexed with lipid preparations will range from about 0.001 mg of protein per 1 ml lipid preparation to about 5 mg of protein per 1 ml lipid preparation.

In some embodiments, the methodology utilized to produce the surfactant composition may include (a) providing a homogeneous lipid preparation of an anionic lipid(s) and/or related compound(s) as described herein (e.g., an unsaturated phosphatidylglycerol, an unsaturated phosphatidylinositol, a saturated short chain phosphatidylglycerol, a saturated short chain phosphatidylinositol, anionic sphingolipid, anionic glycerolipid, unsaturated lyso-PG, saturated lyso-PG, unsaturated lyso-PI, saturated lyso-PI, or a derivative or combination thereof) and (b) adding the preparation of (a) to a preparation of randomly mixed surfactant lipids. The preparation of randomly mixed surfactant lipids may be achieved using any suitable method known in the art. Preferably, the preparation of (a) and/or (b) occurs in an aqueous solution. Most preferably, the preparation is gently mixed to avoid significant fusion or intermixing of lipids between vesicle bilayers in (a) and (b), also as discussed above. In one aspect, the lipids in the preparation of (a) comprise at least 1% of the total lipids in the composition, or any amount from at least 1% to at least 50% or greater, in 1% increments.

Various embodiments of the present invention relate to the use of any of the anionic lipids or related compound formulations described herein, including combinations thereof, to treat and/or prevent inflammation or a pathogen infection, and particularly a coronavirus infection such as one involving SARS-CoV-2, SARS-CoV or MERS-CoV-2 and their variants. The preventative and/or therapeutic methods disclosed herein generally include the administration to an individual (any individual, including infants, children and adults) of any one or more preparations of the anionic lipids and/or related compounds described herein, alone or in combination with other lipids or agents, and/or as a supplement to conventional surfactant preparations or other therapies.

In some embodiments, the methodologies disclosed herein may be utilized to prevent or inhibit inflammation or a pathogen infection associated with particular toll-like receptors, and specifically, one or more of TLR1, TLR2, TLR3, TLR4, TLR6, TLR7, TLR8, TLR9 and/or TLR10. These TLRs have been associated, for example, with various bacterial infections, protozoan and fungal infections, and viral infections. Here, it is to be noted that, while the compositions and methodologies disclosed herein are directed primarily to the treatment or prevent of coronavirus infections, particular instances of such infections may involve other pathogens as well. Accordingly, the compositions and methodologies disclosed herein may be utilized to treat or inhibit inflammation associated with any of these conditions or to prevent or inhibit infection by a pathogen associated with any of these conditions.

Various protocols may be utilized to administer a composition or formulation of the type disclosed herein, and these protocols may relate to the route of administration or the effective amount of a composition or formulation to be administered to an individual. These compositions may be administered in vivo or ex vivo. Suitable in vivo routes of administration may include, but are not limited to, oral, nasal, inhaled, topical, intratracheal, transdermal, rectal, intestinal, intra-luminal, and parenteral routes. Preferred parenteral routes can include, but are not limited to, subcutaneous, intradermal, intravenous, intramuscular, intraarterial, intrathecal and intraperitoneal routes. Preferred topical routes include inhalation by aerosol (i.e., spraying) or topical surface administration to the skin of an animal. Preferably, an agent is administered by nasal, inhaled, intratracheal, topical, or systemic routes (e.g., intraperitoneal, intravenous). Ex vivo refers to performing part of the administration step outside of the patient.

Intravenous, intraperitoneal, and intramuscular administrations of the compositions disclosed herein may be achieved using methods which are known in the art. Aerosol (inhalation) delivery may be performed, for example, using the method described in Stribling et al., Proc. Natl. Acad. Sci. USA 189:11277-11281, 1992, which is incorporated herein by reference in its entirety. Carriers suitable for aerosol delivery have been disclosed herein. Devices for delivery of aerosolized formulations include, but are not limited to, pressurized metered dose inhalers (MDI), dry powder inhalers (DPI), and metered solution devices (MSI), and include devices that are nebulizers and inhalers. Oral delivery may be performed by complexing a therapeutic composition of the present invention to a carrier capable of withstanding degradation by digestive enzymes in the gut of an individual. Examples of such carriers include plastic capsules or tablets, several examples of which are known in the art. Administration of a composition locally within the area of a target cell refers to injecting the composition centimeters and preferably, millimeters from the target cell or tissue.

In human subjects, it known in the art that, using conventional methods for aerosol delivery, only about 10% of the delivered solution typically enters the deep airways, even using an inhaler. Hence, if the aerosolized delivery is by direct inhalation, one may assume a dosage of about 10% of that administered by nebulization methods. Various methods are also known in the art for converting an animal dosage to a human dosage using alometric scaling. For example, essentially, a scale of dosage from mouse to human is based on the clearance ratio of a compound and the body surface of the mouse. The conversion for mg/kg is 1/12th of the “no observed adverse event level” (NO(A)EL) to obtain the concentration for human dosage. This calculation assumes that the elimination between mouse and human is the same.

Preferred amounts of lipid preparations to be delivered to an individual have been disclosed herein. In some embodiments, an effective amount of a preparation of the invention to administer to an individual is an amount that measurably inhibits (or prevents) inflammation or infection by a coronavirus or other pathogen in the individual as compared to in the absence of administration of the formulation. A suitable single dose of a formulation to administer to an individual is a dose that is capable of reducing or preventing at least one symptom, type of injury, or resulting damage, from inflammation or pathogen infection in an individual when administered one or more times over a suitable time period. Preferably, a dose is not toxic to the individual.

It will be appreciated by those skilled in the art that the number of doses of a preparation to be administered to an individual is dependent upon the extent of the inflammatory condition or infection and/or the anticipated or observed physiological damage associated with such inflammation or infection, as well as the response of an individual patient to the treatment. Accordingly, the clinician will be able to determine the appropriate timing for delivery of the formulation in a manner effective to reduce the symptom(s) associated with inflammation or pathogen infection in the individual. Preferably, the agent is delivered within 48 hours, and more preferably 36 hours, and more preferably 24 hours, and more preferably within 12 hours, and more preferably within 6 hours, 5 hours, 4 hours, 3 hours, 2 hours, or 1 hour, or even minutes after the recognition of a condition to be treated by a formulation of the invention; after an event that causes inflammation in an individual or infection of an individual, or that is predicted to cause inflammation in or infection of an individual, which can include administration prior to the development of any symptoms of inflammation or infection in the individual.

Some of the methodologies disclosed herein are primarily intended for use in the prevention and/or treatment of a disease or condition. The term “protecting” may be generically used to convey prevention and/or treatment. Therapeutic compositions of the type disclosed herein, when administered to an individual, may prevent a disease from occurring; cure the disease; delay the onset of the disease; and/or alleviate (reduce, delay, diminish) disease symptoms, signs or causes (e.g., reduce one or more symptoms of the disease; reduce the occurrence of the disease; increase survival of the individual that has or develops the disease; and/or reduce the severity of the disease). As such, some of the compositions and methodologies disclosed herein may be utilized both to prevent disease occurrence (prophylactic treatment) and to treat a subject that has a disease or that is experiencing symptoms of a disease (therapeutic treatment).

Preferred embodiments of the compositions and methodologies disclosed herein are suitable for use in a subject that is a member of the Vertebrate class, Mammalia, including, without limitation, primates, livestock and domestic pets (e.g., a companion animal). Most preferably, the subject will be human. The term “individual” may be interchanged with the term “subject” or “patient” and refers to the subject of a method in accordance with the teachings herein. Accordingly, an individual may include a healthy, normal (non-diseased) individual, but is most typically an individual who has, or is at risk of developing, an inflammatory condition or an infection, especially including a coronavirus infection, or a symptom or indicator thereof as described herein.

Each publication or patent cited herein is incorporated herein by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

This example illustrates the in vitro efficacy of phospholipid compositions of the type described herein in treating corona viruses.

A sample holder containing 96 wells (3 plates) was provided and was plated with VeroE6 cells (a cell lineage isolated from kidney epithelial cells extracted from an African green monkey) at a concentration of 5×10⁴ cells/well in 100 μl of medium. The medium utilized comprised Dulbecco's Modified Eagle Medium (DMEM), 5% fetal bovine serum (FCS), 1% L-glutamine, and 10,000 U/ml penicillin-streptomycin (PS). Here, it is noted that the antibiotics penicillin and streptomycin are commonly utilized to prevent bacterial contamination of cell cultures due to their effective combined action against gram-positive and gram-negative bacteria.

The wells were washed two times with calcium-free and magnesium-free phosphate buffered saline (PBS) (100 μl/well), and then an additional time with a culture medium consisting of DMEM, L-glutamine, 0.1% bovine serum albumin (BSA) and 10,000 U/ml PS.

The cells were then preincubated with lipids in the culture medium (100 μl/well) for 30-60 minutes at 37° C. Samples of SARS-CoV-2 virus were added at multiplicities of infection (MOI) of 0.01 and the virus was allowed to absorb for 2 hours at 37° C. The wells were then washed twice with culture medium at a volume of 100 μl/well. The culture medium was then replaced, with or without lipids, at a volume of 120 μl/well.

At 24 hours and 48 hours, 5 μl of supernatant was collected from each well, for a total of 20 μl of collected supernatant for each condition (virus alone and lipid alone at each concentration), and 60 μl of collected supernatant in total. At 72 hours, supernatant was harvested for each condition.

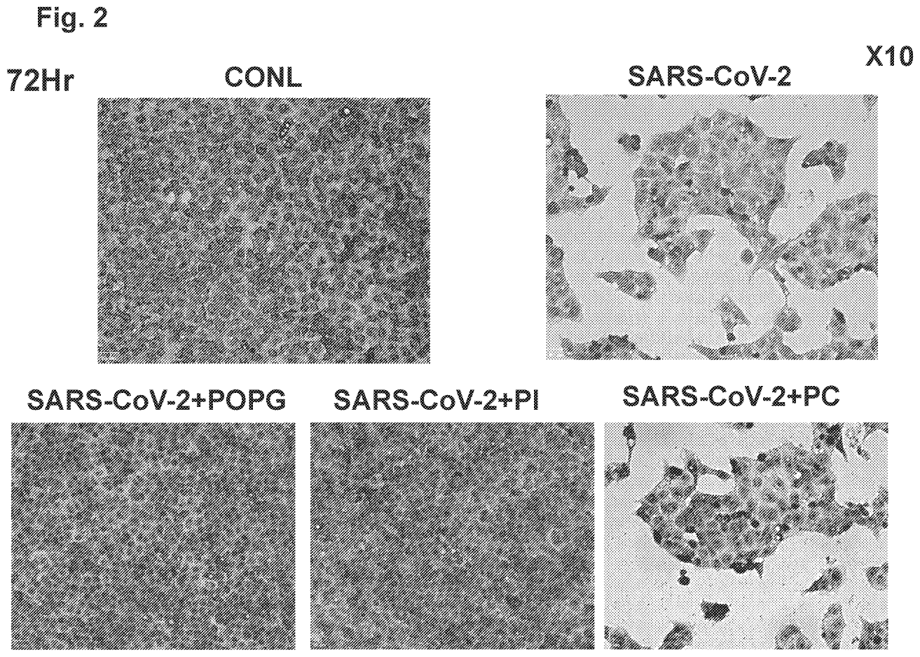

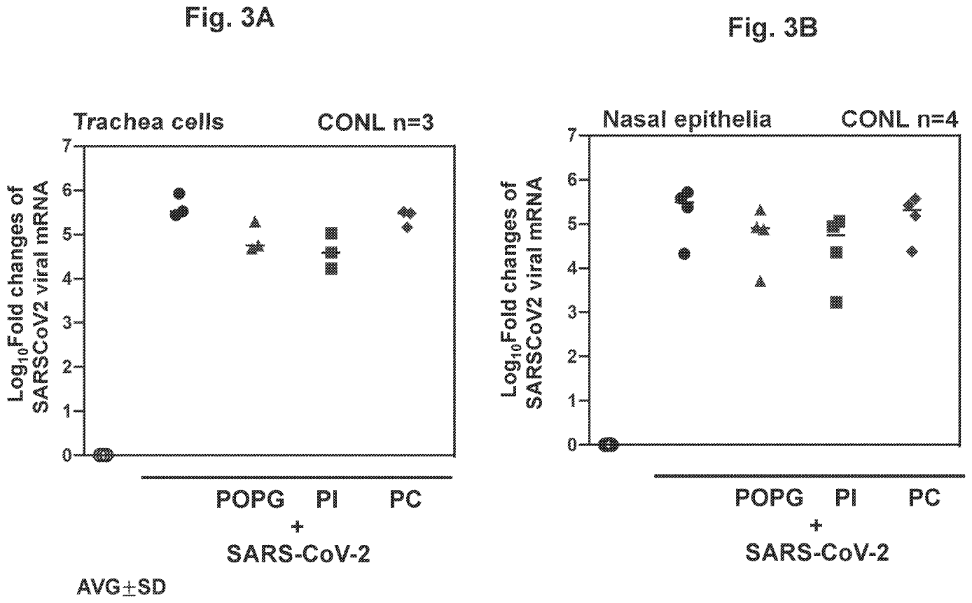

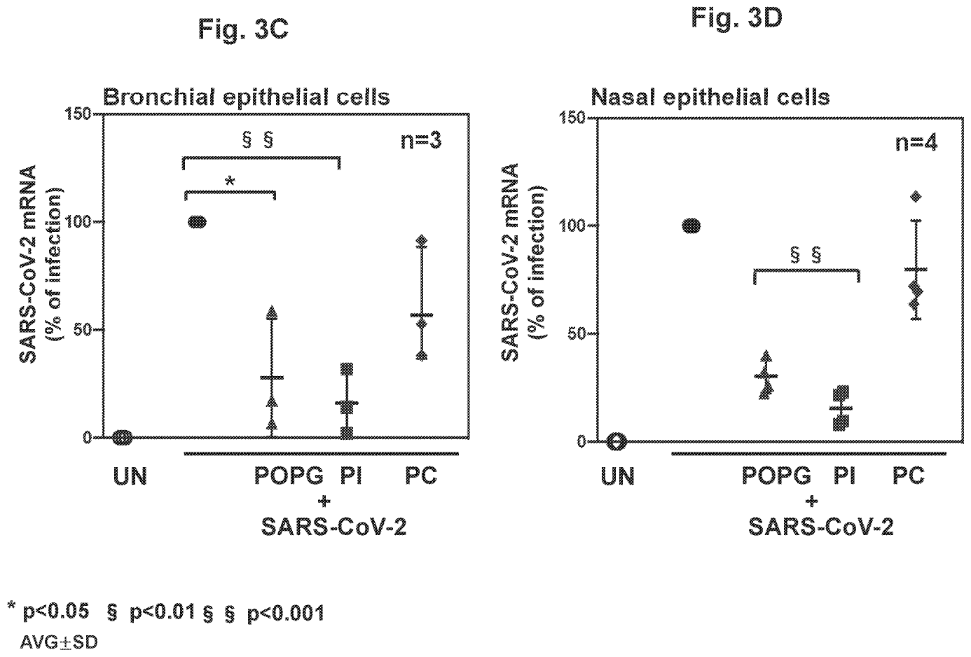

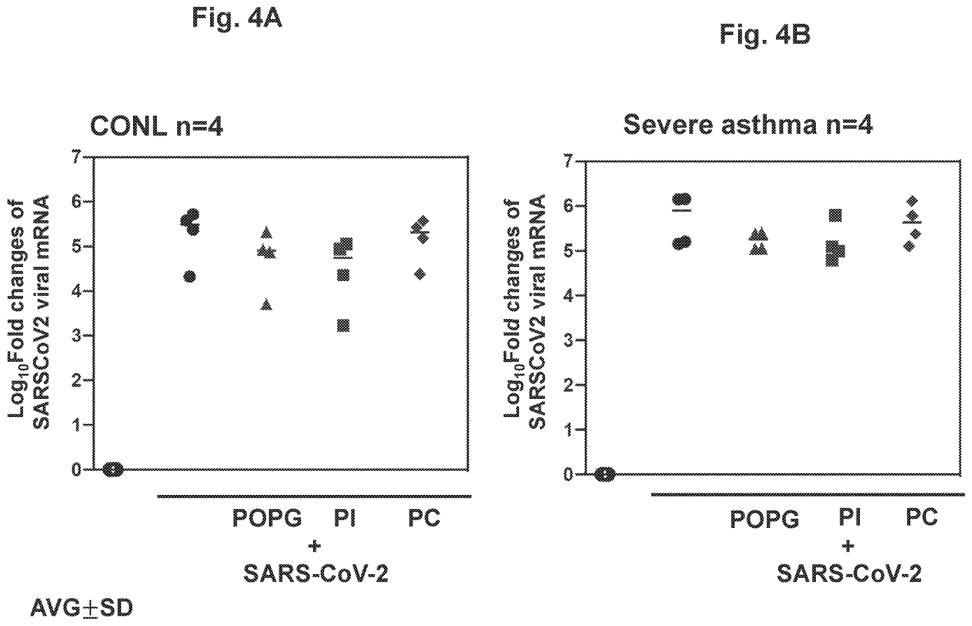

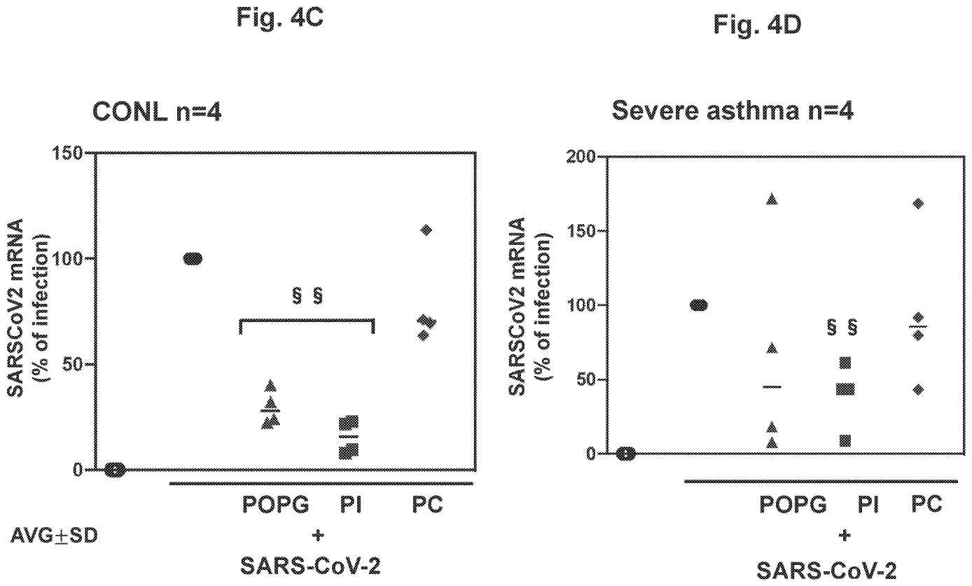

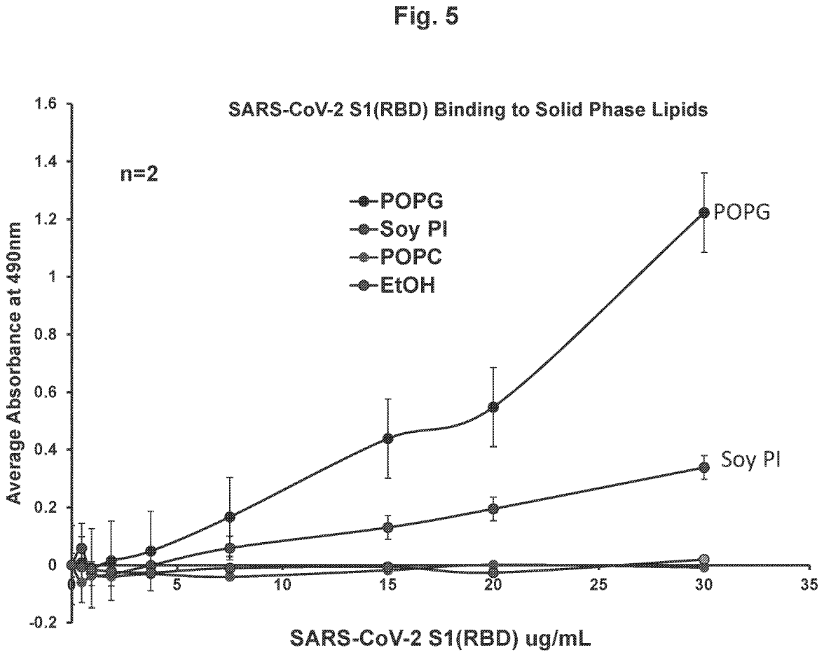

The cells were then washed twice with PBS and fixed with 10% buffered formalin overnight at 4° C. The formalin was then removed with suction, the cells were washed twice with PBS, and stained with hematoxylin and eosin (H&E) stain. Samples of the stained cells are depicted in FIG. 2.

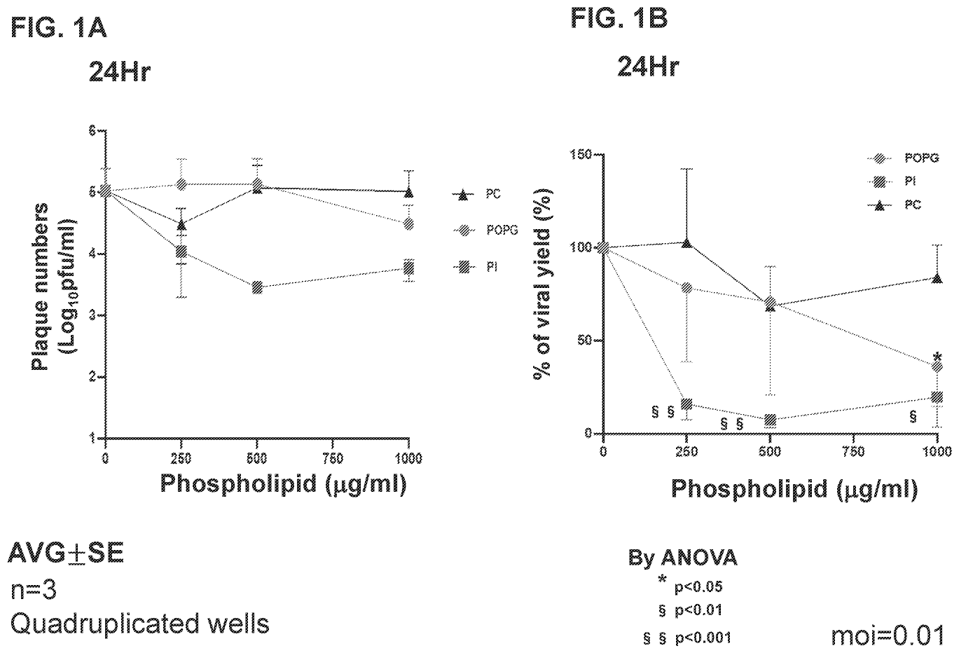

FIGS. 1A and 1B depicts the results obtained for quantitative plaque assay for SARS-CoV-2 (FIG. 1A) and for % viral yield as a function of phospholipid concentration (FIG. 1B).

Simultaneous treatments with virus plus POPG and PI markedly suppressed the viral burden elicited by SARS-CoV-2 in VeroE6 cells by 90% and 99% respectively, at 24 Hr after the initial infection (FIG. 1A). Neither POPG, PI or POPC elicited toxicity at 2 mg/ml on VeroE6 cells as determined by cell viability assays (Numata M, et al. Phosphatidylglycerol suppresses influenza A virus infection. Am J Respir Cell Mol Biol. 2012; 46(4):479-87). As seen therein, in comparison to the control (PC), viral yield decreased significantly for both POPG and PI at concentrations above 500 g/ml, with PI exhibiting significant reduction in viral load at concentrations as low as 250 g/ml.

FIG. 2 illustrates the robust protection provided to the cells by the phospholipids. In particular, the cell cultures for infected cells treated with either POPG and PI remained intact, while the untreated sample and control sample treated with PC both showed significant cytopathic effects.

### Example 2

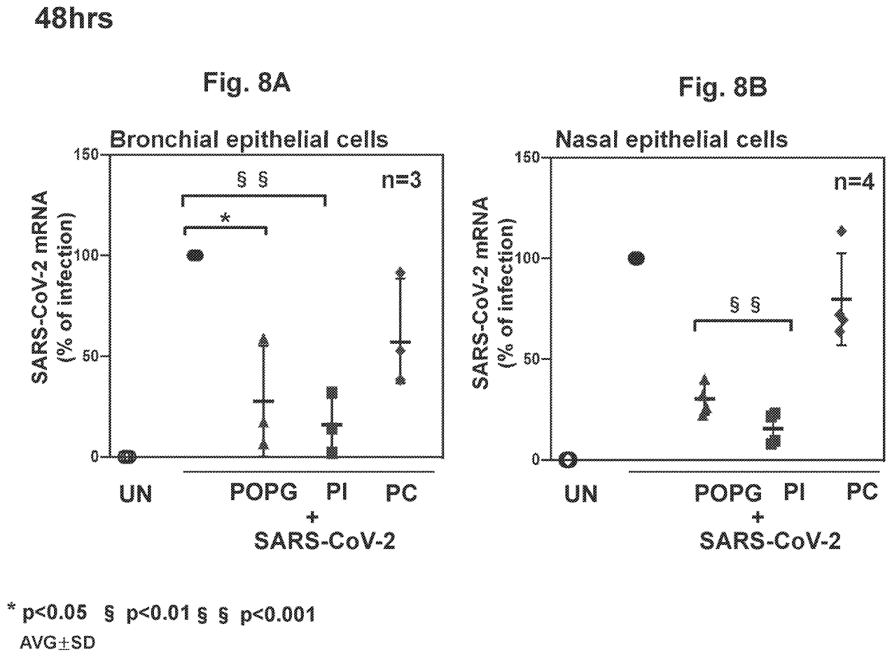

This example demonstrates the anti-viral effect of the lipids against SARS-CoV-2 using primary human airway epithelial cells in ALI cultures. Primary human bronchial or nasal epithelial cells were treated with POPG or PI for 16 hrs before infection, and challenged cells with SARS-CoV-2 at MOI=0.02 for 48 hrs. Cell layers were harvested and RNA-extractions were preformed for qRT-PCR. The prophylaxis treatments with POPG, or PI, markedly reduced SARS-CoV-2 mRNA expression by 75% and 87% respectively using primary cells from multiple healthy control subjects (n=3) (FIGS. 8A and 8B). The control lipid, phosphatidylcholine (PC) did not affect SARS-CoV-2 replication.

### Example 3

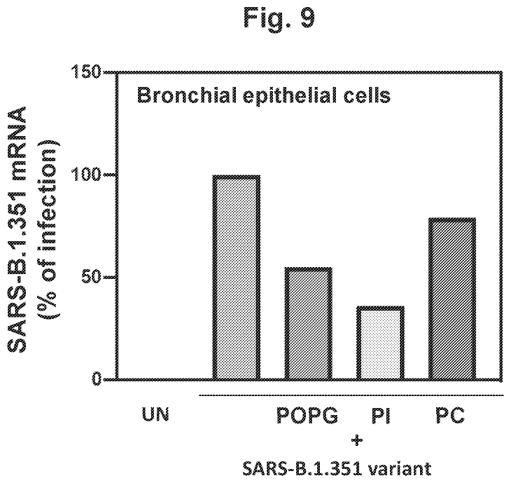

This example demonstrates that both POPG and PI can inhibit SARS-CoV2 variant infection. Differentiated human bronchial epithelial cells were used to determine the antiviral effect of POPG and PI against SARS-CoV-2 variant, B.1.351. The methodology that was stated for the experiments in Example 2 were used. At 48 hrs post infection with B.1.351, cells were harvested and RNA extractions for qRT-PCR were performed. POPG and PI markedly attenuated B.1.351 replication by 50% and 70%, respectively (FIG. 9).

### Example 4

This example demonstrates the concentration-dependent activity of POPG and PI as antagonists of SARS-CoV-2 or its variants. POPG and PI efficacies against SARS-CoV-2 and its variants are examined using various concentrations of the lipids to determine IC50 values. The concentration-dependent activity of POPG and PI as antagonists of SARS-CoV-2, or its variants, are determined by viral burden from culture medium of human primary bronchial and nasal epithelial cells using qRT-PCR and quantitative plaque assays (Runfeng L, et al. Lianhuaqingwen exerts anti-viral and anti-inflammatory activity against novel coronavirus (SARS-CoV-2). Pharmacol Res. 2020; 156:104761.). Cytokine production/secretion (e.g. IL-6, CXCL-10/IP-1) in culture medium is also be measured by ELISA to quantify the inflammatory response. The comparison of IC50 values is used to quantify lipid potency against viruses and determine the rank order potency of each lipid. Primary bronchial/nasal epithelial cells are processed for RNA extraction to quantify the effects of POPG and PI upon the viral replication, viral copy numbers and the cellular anti-viral response by measuring the intracellular anti-viral gene expression pathways by qRT-PCR (Wu X D, et al. The spike protein of severe acute respiratory syndrome (SARS) is cleaved in virus infected Vero-E6 cells. Cell Res. 2004; 14(5):400-6; Runfeng L, et al. Lianhuaqingwen exerts anti-viral and anti-inflammatory activity against novel coronavirus (SARS-CoV-2). Pharmacol Res. 2020; 156:104761.; Prokunina-Olsson L, et al. COVID-19 and emerging viral infections: The case for interferon lambda. J Exp Med. 2020; 217(5)). Further, the therapeutic effectiveness of the lipids is determined by post-infection treatment at 8 hrs and 24 hrs after infection and harvesting supernatant and cells at 48 hours. The infectious particles burden of these cultures is quantified by plaque assays (Wu X D, et al. The spike protein of severe acute respiratory syndrome (SARS) is cleaved in virus infected Vero-E6 cells. Cell Res. 2004; 14(5):400-6; Harcourt J, et al. Severe Acute Respiratory Syndrome Coronavirus 2 from Patient with Coronavirus Disease, United States. Emerg Infect Dis. 2020; 26(6):1266-73). The samples are probed by RNA-seq to elucidate the transcriptional mechanism by which POGP an PI, alter host and viral gene expression after infection with SARS-CoV-2 and its variants.

### Example 5

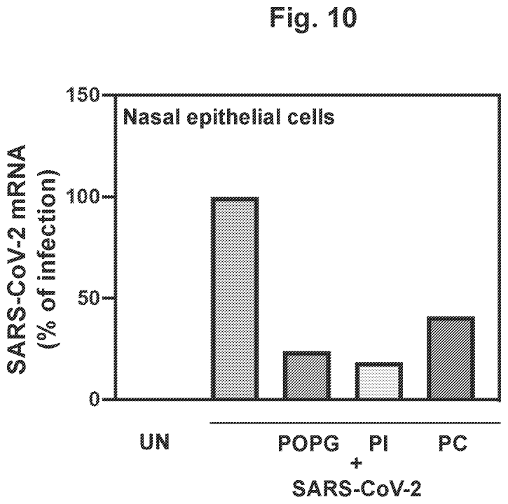

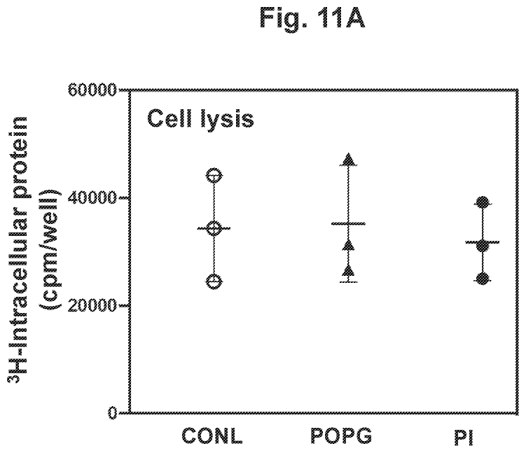

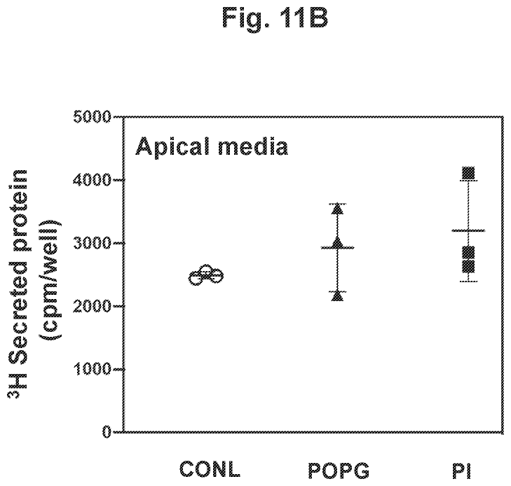

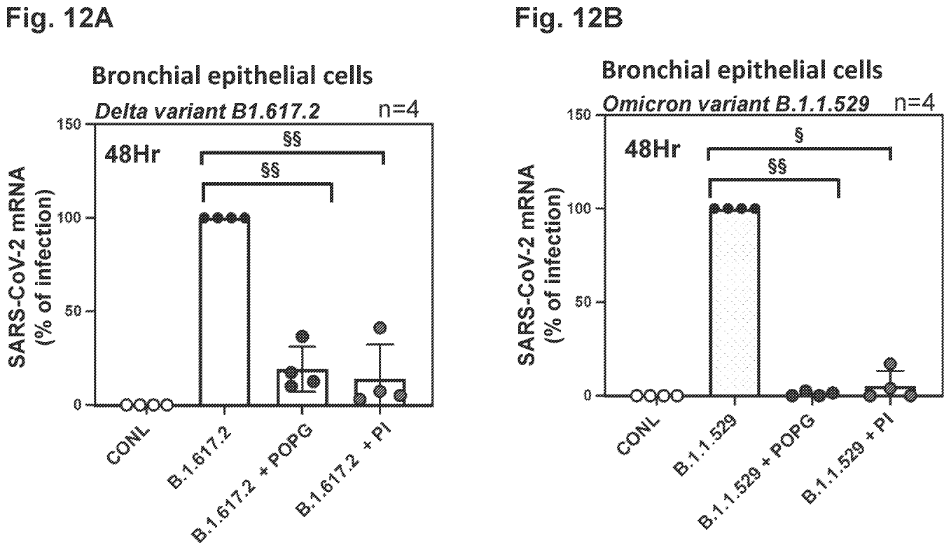

This example demonstrates SARS-CoV-2 variant infections and phospholipid antagonism in highly differentiated and paired primary bronchial and nasal epithelial cells from COPD patients, assessed by qRT-PCR and quantitative plaque assay and probed by RNA-seq. First, POPG and PI were examined to determine if they can inhibit SARS-CoV-2 replication in primary human airway epithelial cells from a patient with COPD. Both POPG and PI strongly attenuated SARS-CoV-2 replication in epithelial cells from a patient with COPD by 80% and 85%, respectively (FIG. 10). Further, it was confirmed that neither POPG nor PI had cytotoxic effects on primary airway epithelial cells from patients with COPD (FIGS. 11A-11B).

Prophylaxis treatments with POPG and PI markedly suppressed the viral replication of SARS-CoV-2 in primary nasal epithelial cells from a patient with COPD, when measured at 48 Hr after the initial infection (FIG. 9). The replication kinetics is examined in differentiated paired human primary nasal epithelial cells and bronchial epithelial cells from patients with COPD and compared to the viral kinetics to the data from Example 4 using control subjects. Using RNAseq, these experiments enable elucidation of the mechanisms by which the lipids inhibit virus-induced inflammatory cytokine production, anti-viral gene expression (Guo K, et al. Interferon resistance of emerging SARS-CoV-2 variants. bioRxiv. 2021. doi: doi.org/10.1101/2021.03.20.436257; Showers W M, et al. Analysis of SARS-CoV-2 mutations over time reveals increasing prevalence of variants in the spike protein and RNA-dependent RNA polymerase. bioRxiv. 2021. doi: doi.org/10.1101/2021.03.05.433666) and mucosal immunity pathways. Viral replication is determined by plaque assays, qRT PCR, and expression of anti-viral genes as previously described in Example 4. Further, the time window is determined for the POPG and PI effects after viral infections are established. ACE2 functions as a receptor for SARS-CoV-2 along with transmembrane protease (TMPRSS2) that is crucial for SARS-CoV-2 cell entry (Sungnak W, et al. SARS-CoV-2 entry factors are highly expressed in nasal epithelial cells together with innate immune genes. Nature Medicine. 2020; 26(5):681-7; Hoffmann M, et al. SARS-CoV-2 Cell Entry Depends on ACE2 and TMPRSS2 and Is Blocked by a Clinically Proven Protease Inhibitor. Cell. 2020; 181(2):271-80 e8). Nasal epithelial cells are a crucial cellular gateway for virus entry and subsequent propagation to expand the viral infection into the gas exchange regions of the lower airway and alveolar compartments (Sungnak W, et al. SARS-CoV-2 entry factors are highly expressed in nasal epithelial cells together with innate immune genes. Nature Medicine. 2020; 26(5):681-7; Peters M C, et al, National Heart L, Blood Institute Severe Asthma Research Program I. COVID-19 Related Genes in Sputum Cells in Asthma: Relationship to Demographic Features and Corticosteroids. Am J Respir Crit Care Med. 2020. Epub 2020/04/30. doi: 10.1164/rccm.202003-08210C. PubMed PMID: 32348692). Interestingly, the use of inhaled corticosteroids (ICS) was associated with lower expression of ACE2 and is useful as a predictor of decreased susceptibility to SARS-CoV-2 infection and decreased COVID19 morbidity (Peters M C, et al. COVID-19-related Genes in Sputum Cells in Asthma. Relationship to Demographic Features and Corticosteroids. Am J Respir Crit Care Med. 2020; 202(1):83-90). There are reports that ACE2 expression is increased in small airways of patients with COPD and this might contribute to the poor outcome of these subjects to COVID19 (Leung J M, et al. COVID-19 and COPD. Eur Respir J. 2020; 56(2)). Both POPG and PI inhibit SARS-CoV-2 and variant replications as shown in FIG. 9 and FIG. 10, the effects of POPG and PI on ACE2, and TRMPRSS2 gene expression is examined, as well.

### Example 6

This example illustrates the in vivo efficacy of phospholipid compositions of the type described herein in treating corona viruses.

A hamster model was utilized to assess the in vivo efficacy of phospholipid compositions of the type described herein in treating a SARS-CoV-2 virus infection. Hamsters have proven to be a valuable animal model for human SARS-CoV-2 infection following intranasal inoculation of SARS-CoV-2 virus. In particular, as with humans, hamsters exhibit (a) the shedding of virus easily detected for several days from oropharyngeal swabs, (b) very high tissue burdens in turbinates and lungs 1-4 days post-inoculation, and (c) the development of severe broncho-interstitial pneumonia. Hence, hamsters provide a useful model for evaluating the protective effect of phospholipids of the type disclosed herein on virus replication and subsequent pulmonary pathology.

Six groups of 6 male Syrian hamsters were acclimated for 5-7 days prior to use and were housed in cages of 3. The treatment applied to each group is depicted in TABLE 1.

TABLE 1
Treatment Protocol

| Group | Intranasal treatment given at time = 0 and +24 hours |
|---|---|
| 1 | 100 ml phosphate buffered saline (PBS) |
| 2 | 100 ml containing 1e4 pfu virus |
| 3 | 100 ml containing mixture of 1e4 pfu virus and 1 mg PI |
| 4 | 100 ml containing mixture of 1e4 pfu virus and 2 mg PI |
| 5 | 100 ml containing 1:1 mixture of PBS and 1 mg PI |
| 6 | 100 ml containing 1:1 mixture of PBS and 2 mg PI |

The hamsters were inoculated under ketamine-xylazine anesthesia. The hamsters were weighed and assessed clinically at the time of inoculation and daily thereafter until necropsy. On days 1, 2, and 3, an oropharyngeal swab was collected from all hamsters in groups 2-4, and the swabs were assayed for the presence of infectious virus. For each group, 3 animals were euthanized and necropsied on day 3, and the remaining 3 were euthanized and necropsied on day 7 post-inoculation. Lung and turbinates from the hamsters in groups 2-4 that were euthanized on day 3 were homogenized and titrated for virus concentration. For all hamsters in the experiment, nasal cavity (turbinates), lung and heart were processed for histopathologic evaluation (2 blocks/hamster and nasal cavity samples require decalcification for sectioning).

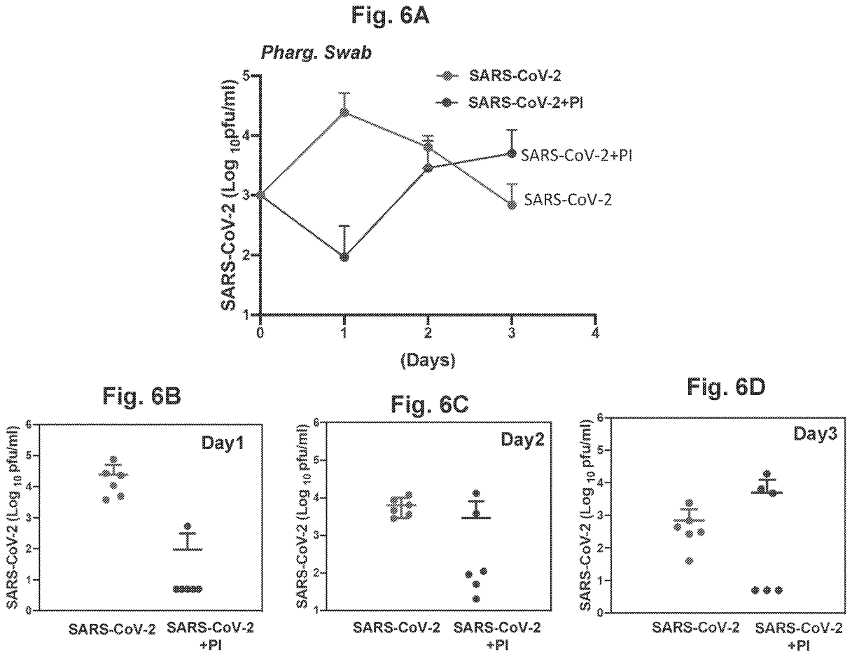

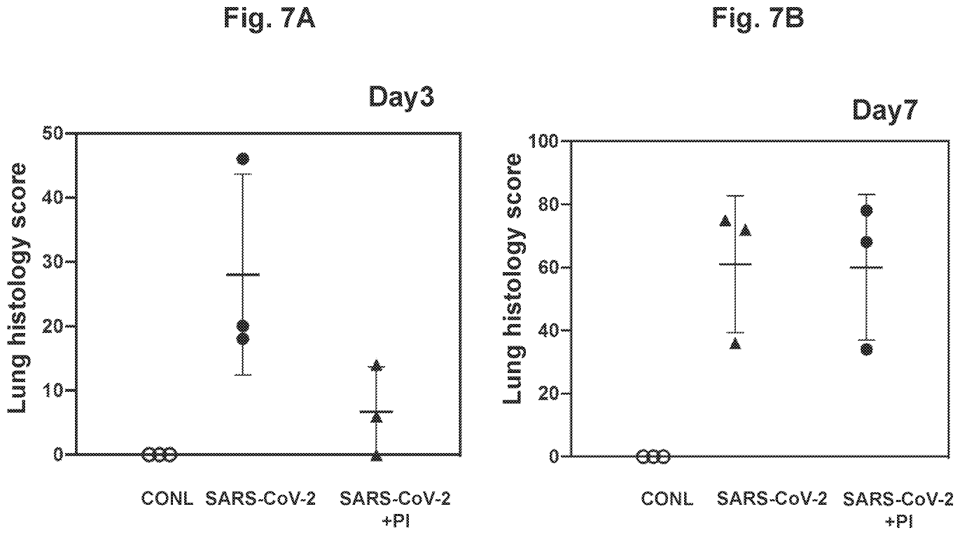

The results are depicted in FIGS. 6A-6D As seen therein, after 24 hours, the viral load in turbinates for the untreated hamsters was significantly higher than the viral load for the hamsters treated with 2 mg of PI. In particular, the untreated group had a mean turbinate viral load of 2.4E+04, while the treated group exhibited a viral load of 9.3E+01. This represents a % inhibition of 99.6% with a single dose of PI.

### Example 7

This example illustrates the efficacy of phospholipids as prophylactic and interventional agents for SARS-CoV-2 in a hamster model.

For groups of hamsters were provided, and were subjected to the treatment specified in TABLE 2.

TABLE 2
Treatment Protocol

| Group | # Hamsters | Treatment | Challenge? |
|---|---|---|---|
| 1 | 8 | 2 mg PI intranasally BID | Yes |
| | | beginning 3 hours pre-virus | |
| | | and repeated at 18 and 42 | |
| | | hours post-virus | |
| 2 | 8 | 2 mg PI intranasally mixed | Yes |
| | | with virus and repeated at | |
| | | 18 and 42 hours post-virus | |
| 3 | 4 | 2 mg PI intranasally BID | No |
| | | beginning 3 hours pre-virus | |
| | | and repeated at 18 and 42 | |
| | | hours post-virus | |
| 4 | 4 | No treatment | Yes |

The hamsters in groups 1-3 were treated with lipid by intranasal instillation as indicated in TABLE 2 under isoflurane anesthesia. Note that group 3 was not challenged, but the timing of PI administration was identical.

The hamsters were weighed and examined clinically once daily from −24 hours to necropsy. Oropharygeal for virus titration swabs were collected once daily on days 1-3 immediately prior to lipid treatment. Half of the animals from each group were euthanized on day 3 and half on day 7; a terminal blood sample was collected from all animals and sera frozen for possible cytokine analysis.

Day 3 necropsies, turbinates and cranial right lobe of the lung were homogenized and titrated for virus. The lung lobe was clamped off prior to excision, and 5 ml of PBS was infused through the trachea, then recovered to obtain brochoalveolar lavage fluid (volume measured). The lavage fluid was clarified by centrifugation at 200-500×g to sediment cells but not the surfactant, and was frozen in multiple vials. The remainder of the lungs and trachea, plus brain and heart, were fixed for histopathology. Day 7 necropsies were performed in a manner identical to those of day 3, except that tissue samples were not homogenized and saved for virus titration.

Serum and BALF were frozen for possible cytokine, bradykinin and coagulopathy analysis. The cell pellet obtained by clarifying BALF was suspended into standard volume and a smear was prepared and stained to obtain a differential count of neutrophils vs. lymphocytes.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims. For convenience, some features of the claimed invention may be set forth separately in specific dependent or independent claims. However, it is to be understood that these features may be combined in various combinations and subcombinations without departing from the scope of the present disclosure. By way of example and not of limitation, the limitations of two or more dependent claims may be combined with each other without departing from the scope of the present disclosure.

What is claimed is:

1. A method of treating or inhibiting a coronavirus infection, comprising administering to an individual who has, or is at risk of developing said coronavirus infection, an amount of at least one anionic lipid or related compound, wherein the amount of the anionic lipid or related compound is effective to inhibit said infection, and wherein the anionic lipid has the following characteristics:
a) has a hydrophobic portion;
b) has a negatively charged portion; and
c) has an uncharged, polar portion.

2. The method of claim 1, wherein the anionic lipid or related compound is selected from the group consisting of: unsaturated phosphatidylglycerol, unsaturated phosphatidylinositol, saturated short chain phosphatidylglycerol, saturated short chain phosphatidylinositol, anionic sphingolipid, anionic glycerolipid, unsaturated lyso-phosphatidylglycerol, saturated lyso-phosphatidylglycerol, unsaturated lyso-phosphatidylinositol, and saturated lyso-phosphatidylinositol, or a derivative thereof.

3. The method of claim 1, wherein the anionic lipid is selected from the group consisting of: an unsaturated phosphatidylglycerol, an unsaturated phosphatidylinositol, a saturated short chain phosphatidylglycerol, and a saturated short chain phosphatidylinositol, or a derivative of the anionic lipid.

4. The method of claim 1, wherein the anionic lipid is unsaturated phosphatidylglycerol, or a derivative thereof.

5. The method of claim 1, wherein the anionic lipid is palmitoyl-oleoyl-phosphatidylglycerol (POPG), or a derivative thereof.

6. The method of claim 1, wherein the anionic lipid is unsaturated phosphatidylinositol, or a derivative thereof.

7. The method of claim 1, wherein the coronavirus infection is associated with a toll-like receptor (TLR) selected from the group consisting of: TLR1, TLR2, TLR3, TLR4, TLR6, TLR7, TLR8, TLR9 and TLR10.

8. The method of claim 1, wherein the anionic lipid or related compound is administered as a composition comprising a homogeneous lipid preparation of the anionic lipid or related compound.

9. The method of claim 1, wherein the anionic lipid or related compound is administered as a composition comprising a preparation of randomly mixed surfactant lipids combined with a homogeneous lipid preparation of the anionic lipid or related compound.

10. The method of claim 1, wherein the anionic lipid or related compound is administered as a compositing comprising a preparation of randomly mixed surfactant lipids, wherein the anionic lipid or related compound comprises at least about 50% of the total lipids in said randomly mixed surfactant lipids.

11. The method of claim 1, wherein the anionic lipid or related compound is administered to the respiratory tract of the individual.

12. The method of claim 1, wherein the anionic lipid is administered to the upper respiratory tract of the subject intranasally or lower respiratory tract via inhalation.

13. The method of claim 1, wherein the anionic lipid is administered intranasally as a formulation containing an emulsifier, a stabilizer and a preservative.

14. The method of claim 1, wherein the coronavirus infection involves at least one virus selected from the group consisting of SARS-CoV-2, SARS-CoV, MERS-CoV and variants thereof.

15. The method of claim 1, wherein the coronavirus infection involves SARS-CoV-2.
